(19) United States
(12) Patent Application Publication
(10) Pub. No.: US 2020/0315937 A1
(43) Pub. Date: Oct. 8, 2020

(54) HAIR TREATMENT AGENT COMPRISING SPECIFIC CHELATING AGENTS

(71) Applicant: Henkel AG & Co. KGaA, Duesseldorf (DE)

(72) Inventor: LUTENDO MAGINA, Centurion (ZA)

(73) Assignee: Henkel AG & Co. KGaA, Duesseldorf (DE)

(21) Appl. No.: 16/756,070

(22) PCT Filed: Dec. 15, 2017
(86) PCT No.: PCT/EP2017/083135

§ 371 Date: Apr. 14, 2020

(51) Int. Cl. A61K 8/44; A61K 8/34; A61Q 5/04

(52) U.S. Cl. CPC A61K 8/44; A61K 8/345; A61K 2800/10; A61K 2800/884; A61K 2800/51; A61Q 5/04

(57) ABSTRACT

The present disclosure relates to a hair treatment agent comprising at least one specific chelating agent, at least one diol and at least one triol. Moreover, the present disclosure relates to the use of the specific chelating agent to remove undesired multivalent metal ions from hair that has previously been treated with relaxing agents containing at least one multivalent metal ion. Furthermore, the present disclosure relates to a method for relaxing keratinic fibers wherein the inventive hair treatment agent is applied after relaxing the hair with a relaxing composition comprising a multivalent metal hydroxide. After the inventive hair treatment agent is applied, the hair is treated with an acidic cleansing composition. Finally, the present disclosure relates to a kit of parts, comprising a hair relaxing composition with a multivalent metal hydroxide, an inventive hair treatment composition and an acidic cleansing composition in separate containers.

### CROSS-REFERENCE TO RELATED APPLICATION

This application is a U.S. National-Stage entry under 35 U.S.C. § 371 based on International Application No. PCT/EP2017/083135, filed Dec. 15, 2017, which was published under PCT Article 21(2), which is incorporated herein by reference in its entirety.

### TECHNICAL FIELD

The present disclosure relates to a hair treatment agent comprising at least one specific chelating agent, at least one diol and at least one triol. Moreover, the present disclosure relates to the use of the specific chelating agent to remove undesired multivalent metal ions from hair that has previously been treated with relaxing agents containing at least one multivalent metal ion. Furthermore, the present disclosure relates to a method for relaxing keratinic fibers wherein the inventive hair treatment agent is applied after relaxing the hair with a relaxing composition comprising a multivalent metal hydroxide. After the inventive hair treatment agent is applied, the hair is treated with an acidic cleansing composition. Finally, the present disclosure relates to a kit of parts, comprising a hair relaxing composition with a multivalent metal hydroxide, an inventive hair treatment composition and an acidic cleansing composition in separate containers.

### BACKGROUND

Commercial chemical hair relaxer products, commonly called “no-lye” relaxers, are two-component products that are mixed together just before use to form the strong organic base, guanidinium hydroxide, in situ. One of the two components typically is an activator solution containing guanidine carbonate and the other component is an emulsion creme containing calcium hydroxide in excess of the stoichiometric amount needed to form the strong organic base. As a result, the mixture contains the byproduct calcium carbonate and unreacted calcium hydroxide, each of which can form an observable deposit of calcium mineral on the surface of the hair.

The adsorption and/or retention of exogenous, multivalent metal ion minerals on hair is undesirable because the deposited mineral can visually dull the appearance of the hair, make the tactile and handling characteristics of the hair heavy, coated, and difficult to style or even lead to hair breakage, and increase its resistance to or interfere with the effectiveness of certain hair treatments, such as dyeing, bleaching, permanent waving or straightening, shampooing, conditioning and the like. Removal of mineral deposit from chemically-relaxed hair presents problems because, following exposure to strong chemical base, the scalp can be vulnerable to irritation and the chemically relaxed hair can be susceptible to weakening damage thereby limiting aggressive removal of mineral ion deposit. In some prior attempts at resolving the mineral buildup problem, chemical pre-treatment products in the form of lotions, shampoos or pre-shampoo sprays have been used to prevent mineral buildup on the hair. These products are applied, sometimes with heat, to the hair before the desired subsequent hair treatment product can be used. However, such chemical pre-treatments are unsuitable for use just before a chemical relaxation procedure because they undesirably increase the risk of predisposing the scalp to burning or irritation from the strong chemical base during the subsequent chemical relaxation process.

The mineral build up can also be prevented if relaxers containing inorganic caustic bases, particularly sodium hydroxide (so-called lye-type relaxers), are used. However, these relaxers tend to have a higher scalp irritation potential as compared to no-lye type hair relaxers. Thus, there is an ongoing need to resolve the problem of adsorption and/or retention of exogenous multivalent metal ions, and of alkaline earth metal ion in particular, on chemically relaxed hair.

### BRIEF SUMMARY

It is thus an object of the present disclosure to provide hair treatment agents, which can be used in combination with hair relaxers, especially no-lye type hair relaxers, and which prevent the undesirable absorption and/or retention of multivalent metal ions, in particular alkaline earth metal ions such as calcium ions, to chemically relaxed hair. It is a further object of the present disclosure to provide a method for relaxing hair resulting in no or reduced buildup of metal ion minerals after relaxing the hair using multivalent metal hydroxides. In an exemplary embodiment, a treatment agent for keratinic fibers, includes in a cosmetically acceptable carrier,

a) at least one chelating agent of formula (I)

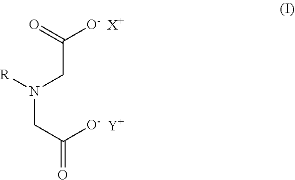

wherein

R is a C₁₋₁₀ alkyl group, a C₁₋₁₀ acyl group or a C₂₋₁₀ dicarbonyl group,

X⁺ and Y⁺ are, independently from each other, physiologically acceptable cations,

b) at least one diol and

c) at least one triol.

Surprisingly, the amount of metal ions, in particular earth metal ions, on hair that has been relaxed with no-lye type relaxers was found to be substantially decreased if a hair treatment agent containing a combination of a specific chelating agent, a diol and a triol is used after chemically relaxing the hair.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first subject matter of the present disclosure is therefore a treatment agent for keratinic fibers, comprising in a cosmetically acceptable carrier,

a) at least one chelating agent of formula (I)

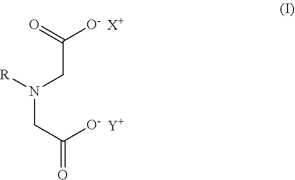

wherein

R is a C₁₋₁₀ alkyl group, a C₁₋₁₀ acyl group or a C₂₋₁₀ dicarbonyl group,

X⁺and Y⁺ are, independently from each other, physiologically acceptable cations,

b) at least one diol and

c) at least one triol.

The chelating agent of formula (I) leads to an effective removal of metal ions, in particular earth metal ions such as calcium ions, which are adhering to the keratinic fibers after chemically relaxing these fibers with no-lye type relaxers, thus resulting in increased hair shine, less hair damage and reduced dryness of hair. These effects can be further improved by combining the at least one chelating agent of formula (I) with at least one diol and at least one triol.

The inventive treatment agent beneficially minimizes the amount of undesirable exogenous multivalent metal ions present in the chemical relaxer composition and which are deposited on the hair during the relaxing process. This leads to increased hair shine as well as decreased hair damage and hair dryness. Still another benefit is that the inventive hair treatment agent can be used directly after relaxing the hair thereby avoiding the need for separate multi step, mineral stripping chemical hair pre-treatments with potentially skin irritating heat and relatively high levels of metal ion chelating agent. Moreover, the inventive treatment composition does not have an adverse effect on the relaxing effect obtained by the chemical relaxer composition, especially no-lye type relaxers.

In principle, keratin fibers shall be understood to mean all animal hair, such as wool, horsehair, angora wool, furs, feathers, and products or textiles produced therefrom. The keratin fibers, however, are preferably human hair.

The term “chelating agent” is used herein to refer to cosmetically acceptable compounds capable of forming water-soluble complexes with multivalent metal ions, especially calcium ions, which are present on the keratinic fibers.

The term “physiologically acceptable cation” as used herein refers to cations including hydrogen, which are physiologically acceptable when topically applied to the skin and/or hair, especially the human skin and/or hair.

The inventive treatment agent contains a cosmetically acceptable carrier. As contemplated herein, the carrier is preferably aqueous, alcoholic or aqueous-alcoholic, especially aqueous. Aqueous carriers contain at least about 30% by wt., especially at least about 50% by wt. of water. Preferred aqueous carriers as contemplated herein therefore contain water in a total amount of from about 30 to about 95% by wt., preferably of from about 40 to about 90% by wt., more preferably of from about 60 to about 90% by wt., especially of from about 80 to about 90% by wt., based on the total weight of the treatment agent.

As a first essential component a), the treatment agent as contemplated herein comprises at least one chelating agent of the formula (I). In formula (I), the residue R can be a C₁₋₁₀ alkyl group. Examples are methyl, ethyl, propyl, isopropyl, butyl, isobutyl, tert-butyl, pentyl, hexyl, heptyl, octyl, nonyl and decyl groups. Moreover, this residue can also be a C₂₋₁₀ dicarbonyl group. Examples are diacetic acid groups, maleic acid groups, fumaric acid groups, succinic acid groups, glutaric acid groups, adipinic acid groups and azelaic acid groups.

Preferred treatment agents contain at least one chelating agent of the formula (I), where R is a C₂₋₈ dicarbonyl group. According to a preferred embodiment of the first subject matter, the residue R in formula (I) is a C₂₋₈ dicarbonyl group, preferably a C₃₋₆ dicarbonyl group.

Chelating agents that are preferably used according to the first subject matter have a certain residue R. It is thus preferred within the scope of the first subject matter if the residue R in formula (I) is a *—CHR¹—(CH₂)ₓ—COO⁻X⁺ group, wherein R¹ is a *—(CH₂)_{y}—COO⁻Y⁺ group, x is a number from 1 to 4, y is a number from 0 to 3 and X⁺ and Y⁺ are, independently from each other, physiological acceptable cations. The symbol * denotes the bond between to residue R and the nitrogen atom of formula (I) or the CH-atom of the residue R and the CH₂-group of residue R¹, respectively. Thus, the residue R is bound via its CHR¹-group to the nitrogen atom of formula (I) and the residue R¹ is bound via its CH₂-group to the CH-group of the residue R.

It is furthermore preferred if X⁺ and Y⁺ in formula (I) each represent a specific physiologically acceptable cation. Within the scope of the first subject matter, it may furthermore be provided that X⁺ and Y⁺ in formula (I) are independently H⁺Li⁺, Na⁺, K⁺ or NH₄⁺, especially Na⁺.

According to a particularly preferred embodiment of the first subject matter, the at least one chelating agent of formula (I) is the tetrasodium salt of N,N-bis(carboxymethyl)-L-glutamic acid. The use of the above-mentioned specific chelating agent in the treatment agents as contemplated herein results in a high removal of earth metal ions, especially calcium ions, from chemically relaxed hair and thus leads to increased hair shine and decreased hair damage and dryness of hair.

Preferred treatment agents according to the first subject matter comprise the at least one chelating agent of formula (I), especially the tetrasodium salt of N,N-bis(carboxymethyl)-L-glutamic acid, in a total amount of from about 0.1 to about 25% by wt., preferably of from about 1 to about 10% by wt., more preferably of from about 1 to about 5% by wt., especially of from about 2 to about 4% by wt., based on the total weight of the hair treatment agent. The use of the above-mentioned amounts of the specific chelating agent of formula (I) result in a good removal of metal earth ions, especially calcium ions, from chemically relaxed hair. In addition, using these amounts ensures that no negative interactions with other ingredients occur, in particular with the at least one diol and triol, which can adversely affect the shelf life and the removing capacity of metal earth salts of the treatment agents as contemplated herein.

As a second essential component b), the treatment agent as contemplated herein comprises at least one diol. The use of this diol in combination with the aforementioned chelating agent of formula (I) further increases the hair shine, the reduced hair damage and hair dryness already achieved by the chelating agent of formula (I).

Particularly good results are obtained within the scope of the first subject matter if the diol is a C₂₋₁₀ alkyl diol, preferably a C₂₋₄ alkyl diol, especially propane-1,2-diol. The combination of the aforementioned specific diols, especially propane-1,2-diol, with the specific chelating agent of formula (I) further increases the hair shine, the reduced hair damage and hair dryness of chemically relaxed hair already achieved by the chelating agent of formula (I).

It is furthermore preferred within the scope of the first subject matter if the agent comprises the at least one diol, especially propane-1,2-diol, in a total amount of from about 0.01 to about 20% by wt., preferably of from about 0.05 to about 10% by wt., more preferably of from about 0.05 to about 2% by wt., especially of from about 0.05 to about 0.4% by wt., based on the total weight of the hair treatment agent. The use of the above-mentioned amounts of the specific diol results in a reduced hair dryness and increased hair shine. In addition, using these amounts ensures that no negative interactions with other ingredients occur, in particular with the chelating agent of formula (I) and the at least one triol, which can adversely affect the shelf life and the capacity to remove metal earth ions of the treatment agent as contemplated herein.

As a third essential component c), the treatment agent as contemplated herein comprises at least one triol. The use of this triol in combination with the aforementioned chelating agent of formula (I) and the at least one diol further increases the hair shine, the reduced hair damage and hair dryness already achieved by the combination of the chelating agent of formula (I) and the at least one diol.

Particularly good results are obtained within the scope of the first subject matter if the triol is a C₂₋₁₀ alkyl triol, preferably a C₂₋₅ alkyl triol, especially glycerin. The combination of the aforementioned specific triols, especially glycerine, with the specific chelating agent of formula (I) and the above cited diols further increases the hair shine, the reduced hair damage and hair dryness of chemically relaxed hair already achieved by the chelating agent of formula (I).

It is furthermore preferred within the scope of the first subject matter if the agent comprises the at least one triol, especially glycerin, in a total amount of from about 0.01 to about 5% by wt., preferably of from about 0.05 to about 4% by wt., more preferably of from about 0.05 to about 2% by wt., especially of from about 0.05 to about 0.4% by wt., based on the total weight of the hair treatment agent. The use of the above-mentioned amounts of the specific triol results in a reduced hair dryness and increased hair shine. In addition, using these amounts ensures that no negative interactions with other ingredients occur, in particular with the chelating agent of formula (I) and the at least one diol, which can adversely affect the shelf life and the capacity to remove metal earth ions of the treatment agent as contemplated herein.

Particularly good results within the scope of the first subject matter are obtained when the at least one diol and the at least one triol are present in the treatment agent in a specific weight ratio. It is therefore preferred, if the agent comprises a weight ratio of the at least one diol b) to the at least one triol c) from about 10:1 to about 1:10, preferably from about 5:1 to about 1:5, more preferably from about 3:1 to about 1:3, especially from about 2:1 to about 1:2. At the above-mentioned weight ratios, the highest increase in hair shine and decrease in hair damage and dryness of hair was observed. Furthermore, due to the absence of unfavorable negative interactions between the specific alcohols and/or the chelating agent of formula (I) at these ratios, a long shelf life as well as good cosmetic properties are obtained.

Within the scope of the present disclosure, it is preferred for the treatment agent to have a pH value from about 4 to about 10, preferably from about 5 to about 10, more preferably from about 5 to about 9, especially from about 5 to about 8. These pH values ensure that there is no irritation of the scalp when the treatment agent is applied to the chemically relaxed hair. Moreover, the high binding capacity of metal earth ions, especially calcium ions, of the at least one chelating agent of formula (I) is not affected in this pH range. The pH value can be set using acids and bases customarily used in hair treatment agents.

In order to prevent adverse effects with respect to the binding capacity of metal earth ions of the at least one chelating agent of formula (I), the treatment agent as contemplated herein preferably does not contain hair dyes and/or oxidizing agents. A preferred embodiment of the first subject matter agent is free from compounds selected from the group of oxidation agents, direct hair dyes, oxidation dyes, oxidation dye precursors and their mixtures. The term “free from” is used herein to refer to agents which contain the aforementioned compounds in a total amount of from 0 to about 0.5% by wt., especially of 0% by wt., based on the total weight of the treatment agent.

Apart from the above mentioned essential components a) to c), the treatment agent can contain further ingredients, selected from the group of (i) surfactants, (ii) vitamins and provitamins, (iii) conditioning agents, and (iv) their mixtures.

So as to intensify the effect as contemplated herein, the inventive treatment agents preferably comprise at least one surfactant, preferably an amphoteric and/or non-ionic surfactant. Surfactants within the meaning of the present disclosure are amphiphilic (bifunctional) compounds, which are composed of at least one hydrophobic molecule part and at least one hydrophilic molecule part. The hydrophobic group is preferably a hydrocarbon chain having 8 to 28 carbon atoms, which can be saturated or unsaturated, linear or branched. This C₈₋₂₈ alkyl chain is particularly preferably linear. The hydrophilic molecule part is preferably a polyethylene oxide group having at least 2 EO units or an acid group. A basic property of the surfactants and emulsifiers is the oriented absorption at interfaces, the aggregation into micelles, and the formation of lyotropic phases.

It has been found to be particularly preferred for the agents as contemplated herein to additionally include at least one non-ionic surfactant. Non-ionic surfactants include a polyol group, a polyalkylene glycol ether group or a combination of a polyol and polyglycol ether group, for example, as the hydrophilic group. Addition products of alkylene oxide to sorbitan esters or saturated linear alcohols and acids with 8 to 30 carbona atoms, each having 2 to 100 moles ethylene oxide per mole of sorbitan ester, alcohol or acid, have proven to be particularly preferred non-ionic surfactants. Cosmetic agents as contemplated herein having excellent properties are obtained if these comprise, as non-ionic surfactants, sorbitan esters with 10 to 30 moles ethylene oxide per mole sorbitan ester in a total amount of from about 0.1 to about 5 wt. %, especially of from about 0.5 to about 4 wt. %, and in particular of from about 1 to about 3 wt. %, based on the total weight of the treatment agent.

Moreover, it is particularly preferred to include at least one amphoteric surfactant. The group of amphoteric or ampholytic surfactants comprises zwitterionic surfactants and ampholytes. These surfactants contain at least one acidic as well as at least one basic hydrophilic group and, depending on the conditions, react acidic or basic. Preferred amphoteric surfactants contain at least one free amino group (basic hydrophilic group) and at least one —COOH or —SO₃H group (acidic hydrophilic group) and can generate inner salts. Examples of suitable amphoteric surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having 8 to 24 carbon atoms in the alkyl group. Particularly preferred ampholytic surfactants are N-cocamidopropyl betaine, cocoacylaminoethylaminopropionate and C₁₂₋₁₈ acylsarcosine. Cosmetic agents as contemplated herein having excellent properties are obtained if these comprise, as amphoteric surfactant, cocamidopropyl betaine in a total amount of from about 1 to about 10 wt. %, especially of from about 2 to about 7 wt. %, and in particular of from about 3 to about 6 wt. %, based on the total weight of the treatment agent.

The treatment agent as contemplated herein can further comprise at least one vitamin or provitamin Preferred vitamins and provitamins are selected from the group of vitamins A, B, C, D, E and K, especially vitamin B₅ or panthenol. It is therefore preferred, if the treatment agent contains panthenol in a total amount of from about 0.1 to about 6 wt. %, especially of from about 0.5 to about 5 wt. %, and in particular of from about 1 to about 4 wt. %, based on the total weight of the treatment agent.

Moreover, the treatment agent according to the present disclosure can comprise at least one conditioning compound to support the effect of the at least one chelating agent of formula (I), the at least one diol and triol with respect to the increased hair shine and decreased hair dryness. Preferred conditioning compounds are selected from silicones and ethoxylated silicones. Cosmetic agents as contemplated herein having excellent properties are obtained if these comprise, as conditioning compounds, at least one ethoxylated silicone with 5 to 15 moles ethylene oxide per mole siloxane in a total amount of from about 0.1 to about 3 wt. %, especially of from about 0.4 to about 2 wt. %, and in particular of from about 0.5 to about 1 wt. %, based on the total weight of the treatment agent.

In the following tables, particularly preferred embodiments E1 to E60 of the treatment agent of the first subject matter are listed (all amounts are given in % by wt., based on the total weight of the treatment agent).

| | E1 | E2 | E3 | E4 |
|---|---|---|---|---|
| Chelating agent formula (I) ¹⁾ | 0.1 to 25 | 1.0 to 10 | 1 to 5 | 2 to 4 |
| Diol | 0.01 to 20 | 0.05 to 10 | 0.05 to 2 | 0.05 to 0.4 |
| Triol | 0.01 to 5 | 0.05 to 4 | 0.05 to 2 | 0.05 to 0.4 |
| Cosmetic carrier | Ad 100 | Ad 100 | Ad 100 | Ad 100 |
| | E5 | E6 | E7 | E8 |
| Chelating agent formula (I) ²⁾ | 0.1 to 25 | 1.0 to 10 | 1 to 5 | 2 to 4 |
| Diol | 0.01 to 20 | 0.05 to 10 | 0.05 to 2 | 0.05 to 0.4 |
| Triol | 0.01 to 5 | 0.05 to 4 | 0.05 to 2 | 0.05 to 0.4 |
| Cosmetic carrier | Ad 100 | Ad 100 | Ad 100 | Ad 100 |
| | E9 | E10 | E11 | E12 |
| Chelating agent formula (I) ³⁾ | 0.1 to 25 | 1.0 to 10 | 1 to 5 | 2 to 4 |
| Diol | 0.01 to 20 | 0.05 to 10 | 0.05 to 2 | 0.05 to 0.4 |
| Triol | 0.01 to 5 | 0.05 to 4 | 0.05 to 2 | 0.05 to 0.4 |
| Cosmetic carrier | Ad 100 | Ad 100 | Ad 100 | Ad 100 |
| | E13 | E14 | E15 | E16 |
| Chelating agent formula (I) ¹⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol | 0.01 to 5 | 0.05 to 4 | 0.05 to 2 | 0.05 to 0.4 |
| Cosmetic carrier | Ad 100 | Ad 100 | Ad 100 | Ad 100 |
| | E17 | E18 | E19 | E20 |
| Chelating agent formula (I) ²⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol | 0.01 to 5 | 0.05 to 4 | 0.05 to 2 | 0.05 to 0.4 |
| Cosmetic carrier | Ad 100 | Ad 100 | Ad 100 | Ad 100 |
| | E21 | E22 | E23 | E24 |
| Chelating agent formula (I) ³⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol | 0.01 to 5 | 0.05 to 4 | 0.05 to 2 | 0.05 to 0.4 |
| Cosmetic carrier | Ad 100 | Ad 100 | Ad 100 | Ad 100 |
| | E25 | E26 | E27 | E28 |
| Chelating agent formula (I) ¹⁾ | 0.1 to 25 | 1.0 to 10 | 1 to 5 | 2 to 4 |
| Diol | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Cosmetic carrier | Ad 100 | Ad 100 | Ad 100 | Ad 100 |
| | E29 | E30 | E31 | E32 |
| Chelating agent formula (I) ²⁾ | 0.1 to 25 | 1.0 to 10 | 1 to 5 | 2 to 4 |
| Diol | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Cosmetic carrier | Ad 100 | Ad 100 | Ad 100 | Ad 100 |
| | E33 | E34 | E35 | E36 |
| Chelating agent formula (I) ³⁾ | 0.1 to 25 | 1.0 to 10 | 1 to 5 | 2 to 4 |
| Diol | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Cosmetic carrier | Ad 100 | Ad 100 | Ad 100 | Ad 100 |
| | E37 | E38 | E39 | E40 |
| Chelating agent formula (I) ¹⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Cosmetic carrier | Ad 100 | Ad 100 | Ad 100 | Ad 100 |
| | E41 | E42 | E43 | E44 |
| Chelating agent formula (I) ²⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Cosmetic carrier | Ad 100 | Ad 100 | Ad 100 | Ad 100 |
| | E45 | E46 | E47 | E48 |
| Chelating agent formula (I) ³⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Cosmetic carrier | Ad 100 | Ad 100 | Ad 100 | Ad 100 |
| | E49 | E50 | E51 | E52 |
| Chelating agent formula (I) ¹⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Amphoteric surfactant ⁶⁾ | 1 to 10 | 1.5 to 8 | 2 to 7 | 3 to 6 |
| Panthenol | 0.1 to 6 | 0.3 to 5.5 | 0.5 to 5 | 1 to 4 |
| Conditioning agent ⁷⁾ | 0.1 to 3 | 0.2 to 2.5 | 0.4 to 2 | 0.5 to 1 |
| Cosmetic carrier | Ad 100 | Ad 100 | Ad 100 | Ad 100 |
| | E53 | E54 | E55 | E56 |
| Chelating agent formula (I) ²⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Amphoteric surfactant ⁶⁾ | 1 to 10 | 1.5 to 8 | 2 to 7 | 3 to 6 |
| Panthenol | 0.1 to 6 | 0.3 to 5.5 | 0.5 to 5 | 1 to 4 |
| Conditioning agent ⁷⁾ | 0.1 to 3 | 0.2 to 2.5 | 0.4 to 2 | 0.5 to 1 |
| Cosmetic carrier | Ad 100 | Ad 100 | Ad 100 | Ad 100 |
| | E57 | E58 | E59 | E60 |
| Chelating agent formula (I) ³⁾ | 0.1 to 25 | 1.0 to 10 | 1 to 5 | 2 to 4 |
| Diol ⁴⁾ | 0.01 to 20 | 0.05 to 10 | 0.05 to 2 | 0.05 to 0.4 |
| Triol ⁵⁾ | 0.01 to 5 | 0.05 to 4 | 0.05 to 2 | 0.05 to 0.4 |
| Amphoteric surfactant ⁶⁾ | 1 to 10 | 1.5 to 8 | 2 to 7 | 3 to 6 |
| Panthenol | 0.1 to 6 | 0.3 to 5.5 | 0.5 to 5 | 1 to 4 |
| Conditioning agent ⁷⁾ | 0.1 to 3 | 0.2 to 2.5 | 0.4 to 2 | 0.5 to 1 |
| Cosmetic carrier | Ad 100 | Ad 100 | Ad 100 | Ad 100 |

¹⁾ R is a C₁₋₁₀ alkyl group, a C₁₋₁₀ acyl group or a C₂₋₁₀ dicarbonyl group, X⁺ and Y⁺ are, independently from each other, physiologically acceptable cations,
²⁾ R is a *—CHR¹—(CH₂)ₓ—COO⁻X⁺ group, wherein R¹ is a *—(CH₂)_{y}—COO⁻Y⁺ group, x is a number from 1 to 4 and y is a number from 0 to 3, X⁺ and Y⁺ are, independently from each other, Na⁺,
³⁾ tetrasodium salt of N,N-bis(carboxymethyl)-L-glutamic acid,
⁴⁾ propane-1,2-diol,
⁵⁾ glycerin
⁶⁾ cocamidopropyl betaine
⁷⁾ ethoxylated siloxane with 12 moles ethylene oxide per mole siloxane.

The afore listed preferred embodiments E1 to E60 of the inventive treatment agent effectively remove calcium ions from chemically relaxed hair and thus result in increased hair shine and decreased hair damage and dryness of hair. Moreover, these agents do not lead to scalp irritation as the pre-treatment agents of the state of the art. Since these agents effectively remove undesirable buildup of metal earth ions after chemically relaxing the hair, they render it possible to use less irritating no-lye relaxers while at the same time obtaining relaxed hair having the same or even better cosmetic properties as compared to lye-relaxers, which do not lead to undesirable buildup of metal ions. Additionally, higher amounts of multivalent metal hydroxide can be used in the relaxing composition thus leading to excellent relaxing results and cosmetic properties for hair, where higher amounts of relaxer are necessary.

A second subject matter of the present disclosure is the use of a chelating agent of formula (I)

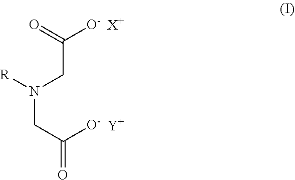

wherein

R is a C₁₋₁₀ alkyl group, a C₁₋₁₀ acyl group or a C₂₋₁₀ dicarbonyl group, and

X⁺ and Y⁺ are, independently from each other, physiologically acceptable cations, to remove calcium ions from hair, especially from hair that has previously been treated with relaxing agents containing calcium hydroxide.

These chelating agents, especially the preferred chelating agents of formula (I) described in the first subject matter, show excellent calcium ion binding capacity and thus lead to an effective removal of excess calcium ions from chemically relaxed hair, especially hair relaxed with no-lye type relaxers containing calcium hydroxide and an activator, for example guanidine carbonate. Thus, the undesirable buildup of calcium ions leading to decreased shine, hair damage and dryness of hair, can effectively be prevented by using chelating agents of the formula (I).

Preferably, the chelating agents of formula (I), especially the tetrasodium salt of N,N-bis(carboxymethyl)-L-glutamic acid, are used in specific amounts in order to maximize the amount of calcium ions that are removed from the chemically treated hair. A preferred embodiment of this subject matter least one chelating agent of formula (I), especially the tetrasodium salt of N,N-bis(carboxymethyl)-L-glutamic acid, is used in a total amount of from about 0.1 to about 25% by wt., preferably of from about 1.0 to about 10% by wt., more preferably of from about 1 to about 5% by wt., especially of from about 2 to about 4% by wt., based on the total weight of the treatment agent.

What was said above with respect to the treatment agents, especially the preferred chelating agents of formula (I), as contemplated herein applies, mutatis mutandis, with respect to preferred further embodiments of the use as contemplated herein.

The treatment agent of the first subject matter is preferably used in a method for relaxing keratinic fibers, especially hair. Thus, a third subject matter of the present disclosure is a method for relaxing keratinic fibers, the method comprising the sequential steps of:

a) contacting keratinic fibers, especially hair, with a hair relaxing composition (A1) containing at least one multivalent metal hydroxide base in an amount sufficient to relax at least part of the keratinic fibers for a period of from about 1 to about 20 minutes, especially for from about 3 to about 5 minutes, and rising the composition (A1) from the hair to provide alkaline, chemically-relaxed hair;
b) contacting the alkaline, chemically-relaxed hair of step a) with an inventive treatment agent (A2) for a period of from about 1 to about 20 minutes, especially for from about 3 to about 5 minutes, and rinsing the composition (A2) from the hair; and
c) contacting the chemically-relaxed hair of step b) with an aqueous cleansing agent (A3) containing at least one surfactant and at least one acid and having a pH from about 1 to about 5 for a period of from about 1 to about 20 minutes, especially for from about 3 to about 5 minutes, rinsing the composition (A3) from the hair and optionally drying the hair.

The inventive method leads to relaxed hair with less buildup of undesirable calcium ions and calcium salts arising from the relaxing step a) because these ions and salts are effectively removed by using the inventive treatment composition (A2) in combination with the neutralizing composition (A3). Thus, the amount of calcium ions and salts after inventive steps b) and c) have been performed is less than the amount of calcium ions and salts if either step b) and/or step c) is omitted. Due to less undesirable build up, the method results in increased hair shine, less hair damage and reduced dryness of hair. Additionally, the inventive method provides a resolution of the problem of deposition of alkaline earth metal ions and salts on chemically-relaxed hair, thereby avoiding the need for separate multi step, mineral stripping chemical hair pre-treatments with potentially skin irritating heat and relatively high levels of metal ion chelating agent. Additionally, higher amounts of multivalent metal hydroxide can be used in the relaxing composition thus leading to excellent relaxing results and cosmetic properties for hair, where higher amounts of relaxer are necessary.

The term “alkaline, chemically-relaxed hair” as used herein refers to naturally curly hair which has had its natural curl relaxed or straightened by contact with a chemical relaxer composition, preferably a no-lye type chemical relaxer composition comprising calcium hydroxide and guanidine carbonate as activator.

According to a preferred embodiment of the third subject matter, a specific multivalent metal hydroxide base is used in step a) of the inventive process. It is therefore preferred, if calcium hydroxide is used as the at least one multivalent metal hydroxide base in composition (A1). The inventive process renders it possible to use less irritating no-lye type relaxers without the problem of undesired metal ion build up after the relaxing step a).

A preferred process is exemplified in that specific amounts of the multivalent metal hydroxide are used in step a) of the inventive process. A preferred embodiment of this subject matter composition (A1) comprises the at least one multivalent metal hydroxide, especially calcium hydroxide, base in a total amount of from about 10 to about 40% by wt., preferably of from about 15 to about 35% by wt., more preferably of from about 20 to about 30% by wt., especially of from about 23 to about 28% by wt., based on the total weight of composition (Al). The use of the aforementioned amounts of metal hydroxide, especially calcium hydroxide, lead to a sufficient relaxing of the hair in step a) of the inventive process. Due to the removal of metal ion and metal salt build up in steps b) and c) of the inventive process, higher amounts of metal hydroxide base can be used, thus allowing to obtain excellent relaxing results independent of the hair type.

Particularly good results are obtained within the scope of this subject matter if the composition (A1) further comprises the at least one guanidine salt, preferably guanidine carbonate, in a total amount of from about 0.2 to about 6% by wt., preferably of from about 0.3 to about 5% by wt., more preferably of from about 0.4 to about 4% by wt., especially of from about 0.5 to about 3% by wt., based on the total weight of composition (Al). This guanidinium salt reacts with the at least one multivalent metal hydroxide, especially calcium hydroxide, to form the relaxing component guanidinium hydroxide and the by-product calcium carbonate. Since the at least one multivalent metal hydroxide, especially calcium hydroxide, is used in excess, the composition (A1) comprises reactive guanidinium hydroxide, calcium carbonate and calcium hydroxide. The formed calcium salts and ions are effectively removed during steps b) and c) of the inventive process, so that excellent relaxing results as well as increased hair shine, less hair damage and reduced dryness of hair is obtained with the inventive process.

The composition (A1) used in step a) of the inventive process is preferably prepared by mixing a composition (A1a) containing the at least one multivalent metal hydroxide, especially calcium hydroxide, with a composition (A1b) containing the at least one guanidinium salt prior to application of the composition (A1) to the hair. Composition (A1a) is preferably mixed with composition (A1b) in a weight ratio of from about 1:4 to about 4:1, especially of about 4:1.

The composition (A1) preferably comprises water as the carrier material. Preferred compositions (A1) comprise water in a total amount of from about 30 to about 75% by wt., preferably of from about 35 to about 70% by wt., more preferably of from about 40 to about 65% by wt., especially of from about 45 to about 60% by wt., based on the total weight of composition (A1). The composition (A1) can also comprise further components, selected from the group of (i) oils, (ii) surfactants, (iii) CS-Cm alcohols, (iv) conditioning compounds, (v) thickening agents, and (vi) mixtures of the aforementioned components.

Preferred oils are selected from the group of mineral oils, petrolatum, vegetable oils, animal oils and their mixtures. Especially preferred oils are selected from petrolatum, mineral oil, almond oil, seed extract oil of hordeum vulagre and their mixtures. The at least one oil is preferably present in a total amount of from about 10 to about 40% by wt., preferably of from about 12 to about 30% by wt., more preferably of from about 15 to about 25% by wt., especially of from about 18 to about 23% by wt., based on the total weight of composition (A1).

Preferred surfactants are selected from the group of non-ionic surfactants, especially ethoxylated C₈-C₃₀ alcohols containing from about 5 to about 30 moles of ethylene oxide per mole alcohol. The at least one surfactant is preferably present in a total amount of from about 0.5 to about 10% by wt., preferably of from about 1 to about 6% by wt., more preferably of from about 1.5 to about 5% by wt., especially of from about 2 to about 4% by wt., based on the total weight of composition (A1).

The C₈-C₃₀ alcohols, preferably cetearyl alcohol, are present in a total amount of from about 1 to about 15% by wt., preferably of from about 2 to about 10% by wt., more preferably of from about 3 to about 9% by wt., especially of from about 4 to about 8% by wt., based on the total weight of composition (A1).

Preferred conditioning compounds are selected from the group of protein hydrolysates, vitamins, provitamins and their mixtures. Especially preferred conditioning agents are protein hydrolysates and panthenol. The at least one conditioning compound is preferably present in a total amount of from about 0.2 to about 2% by wt., preferably of from about 0.3 to about 3% by wt., more preferably of from about 0.4 to about 2% by wt., especially of from about 0.5 to about 1% by wt., based on the total weight of composition (A1).

Suitable thickeners include organic polymers such as polyacrylic acid, hydroxypropyl cellulose and hydroxyethylcellulose, anhydrous aluminum silicate, hydrated magnesium aluminum silicate and colloidal clays. A preferred thickener is polyacrylic acid. The thickener is preferably present in a total amount of from about 0.1 to about 1.5% by wt., preferably of from about 0.1 to about 1% by wt., more preferably of from about 0.1 to about 0.8% by wt., especially of from about 0.2 to about 0.6% by wt., based on the total weight of composition (A1).

A particularly preferred composition (A1) additionally comprises—based on the total weight of composition (A1):
from about 45 to about 60% by wt. of water,
from about 18 to about 23% by wt. of at least one oil, preferably of a mixture of petrolatum, mineral oil, almond oil and hordeum vulgare seed oil,
from about 2 to about 4% by wt. of at least one non-ionic surfactant, preferably a mixture of a C₁₆-C₁₈ alcohol with 10 to 20 moles of ethylene oxide per mole alcohol,
from about 4 to about 8% by wt. of at least one C₈-C₃₀ alcohol, preferably cetearyl alcohol,
from about 0.5 to about 1% by wt. of at least one conditioning compound, preferably a mixture of hydrolyzed protein and panthenol, and
from about 0.2 to about 0.6% by wt. of at least one thickener, preferably polyacrylic acid.

In step b) of the inventive process, the relaxed hair is contacted with the inventive treatment agent (A2) of the first subject matter. This treatment leads to effective removal of multivalent metal ions which are present on the relaxed hair, thus resulting in increased hair shine, less hair damage and reduced dryness of hair. With respect to the composition (A2) used in step b) of the inventive process, reference is made to the first subject matter of the present disclosure.

In step c) of the inventive process, an acidic treatment composition (A3) is used to remove multivalent metal salts present on the relaxed hair after application of the composition (A1) in step a) of the inventive process so that the good cosmetic properties achieved after step b) are further increased. In principle, all cosmetically acceptable acids can be used in composition (A3). However, the use of specific acids is favorable in terms of removal of multivalent metal salts. A preferred embodiment of the third subject matter at least one acid in composition (A3) is selected from the group of acetic acid, lactic acid, citric acid and their mixtures, preferably from lactic acid. The use of lactic acid in composition (A3) has been proven to be especially effective in removing multivalent metal salts on chemically relaxed hairs, thus leading to increased hair shine, less hair damage and reduced dryness of hair.

Within the scope of the third subject matter, it is preferred if the at least one acid in composition (A3) is present in a total amount of from about 0.1 to about 25% by wt., preferably of from about 0.1 to about 5% by wt., more preferably of from about 0.5 to about 4% by wt., especially of from about 0.5 to about 2% by wt., based on the total weight of composition (A3). The aforementioned amounts of the at least one acid, especially lactic acid, have been proven to effectively remove the multivalent metal salts still present on relaxed hair after steps a) and b) of the inventive method. Moreover, these amounts lead to a closing of the hair cuticula which has been opened by the highly basic relaxing composition (A1). Thus, the good cosmetic properties of relaxed hair obtained after steps a) and b) can be further improved.

In order to allow a sufficient wetting of the relaxed hair with the cleaning composition (A3), this composition comprises at least one surfactant. It is preferred if the at least one surfactant in composition (A3) is selected from anionic surfactants and is present in a total amount from about 2.5 to about 12% by wt., preferably from about 3 to about 9% by wt., more preferably from about 3.5 to about 8% by wt., especially from about 4 to about 7% by wt., based on the total weight of composition (A3). Suitable anionic surfactants are the alkyl sulphates, alkyl ether sulphates, alkaryl sulphonates, alkyl succinates, alkyl sulphosuccinates, N-alkoyl sarcosinates, and alpha-olefin sulphonates, especially their sodium, magnesium, ammonium and mono-, di- and triethanolamine salts. The alkyl groups generally contain from 8 to 18 carbon atoms and may be unsaturated. The alkyl ether sulphates may contain from one to 10 ethylene oxide or propylene oxide units per molecule, and preferably contain 2 to 3 ethylene oxide units per molecule.

The composition (A3) is preferably in the form of a shampoo and can comprise further components, selected from the group of (i) surfactants, (ii) conditioning compounds, (iii) thickening agents, and (vi) mixtures of the aforementioned components. The composition (A3) is preferably an aqueous composition comprising water as carrier component. Preferred compositions (A3) comprise water in a total amount of from about 40 to about 95% by wt., preferably of from about 45 to about 90% by wt., more preferably of from about 50 to about 85% by wt., especially of from about 55 to about 80% by wt., based on the total weight of composition (A1).

Cationic surfactants are quaternary ammonium surfactants and amine surfactants that are permanently positively charged at the pH of the composition (A3) and contain at least one or more nonionic hydrophilic moieties. Preferred cationic surfactants are those which are useful for providing conditioning benefits, particularly hair conditioning benefits and which are quaternary ammonium or amino compounds having at least one N-radical containing one or more hydrophilic moieties selected from alkoxy, polyoxyalkylene, alkylamido, hydroxyalkyl, and alkylester moieties, and combinations thereof. Suitable nonionic surfactants include mono or di alkyl alkanolamides or alkyl polyglucosides. Examples include coco mono or diethanolamide, coco mono isopropanolamide, and coco di glucoside. The amphoteric surfactants suitable for use in the composition (A3) may include alkyl amine oxides, alkyl betaines, alkyl amidopropyl betaines, alkyl mono- or dialkanolamides, alkyl sulphobetaines, alkyl glycinates and alkyl carboxyglycinates, wherein the alkyl groups have from 8 to 18 carbon atoms. Examples include sodium lauroamphoacetate, lauryl amine oxide, cocomonoethanolamide, cocodiethanolamide, cocamidopropyl betaine, cocodimethyl sulphopropyl betaine and preferably cocobetaine. The at least one surfactant, especially a mixture of cationic, nonionic and amphoteric surfactants, is present in the composition (A3) in a total amount of from about 5 to about 40% by weight, preferably of from about 15 to about 30% by wt., based on the total weight of the composition (A3).

Suitable conditioning compounds are selected from cationic polymers, especially cationic derivatives of a guar gum and hydroxyethyl cellulose. Cationic guar gum derivatives are those given the CTFA designation guar hydroxypropyl trimonium chloride and are available commercially under the trademark JAGUAR. Cationic hydroxyethyl cellulose is known under the CTFA designation polyquaternium-10. Further suitable conditioning compounds are selected from silicones, especially polyalkyl siloxanes, ethoxylated polyalkyl siloxanes with 10 to 20 moles of ethylene oxide per mole siloxane or mixtures thereof. Polyalkyl siloxanes include polydimethyl siloxanes which have the CTFA designation dimethicone, having a viscosity of from about 5 to about 100,000 centistokes at about 25° C. Moreover, vitamins and provitamins, like panthenol, can be included as conditioning agents. The at least one conditioning agent, especially a mixture of cationic guar gum, cationic hydroxyethyl cellulose, polydimethyl siloxane and polydimethyl siloxane with 15 moles ethylene oxide per mole polydimethyl siloxane, is present in the composition (A3) in a total amount of from about 1 to about 10% by weight, preferably of from about 2 to about 5% by wt., based on the total weight of the composition (A3).

Suitable thickening agents are organic polymers, for example polyacrylates, and electrolytes, especially sodium chloride. The at least one thickener, especially sodium chloride, is preferably present in a total amount of from about 1 to about 10% by weight, preferably from about 3 to about 6% by wt., based on the total weight of the composition (A3).

A particularly preferred composition (A3) additionally comprises - based on the total weight of composition (A3):
from about 55 to about 80% by wt. of water,
from about 15 to about 30% by wt. of at least one surfactant, preferably of a mixture behentrimonium chloride, coco mono ethanolamine, sodium lauroamphoacetate and sodium trideceth sulfate,
from about 2 to about 5 by wt. of at least one conditioning agent, preferably a mixture of a cationic guar gum, a cationic hydroxyethyl cellulose, polydimethyl siloxane and polydimethyl siloxane containing 15 moles ethylene oxide per mole polydimethyl siloxane, and
from about 3 to about 6% by wt. of at least one thickener, preferably sodium chloride.

In order to increase the cosmetic properties of the relaxed hair, it is preferred if a conditioning composition (A4) is applied after performing step c) of the inventive method. In order to fully remove any remaining multivalent metal salts from the relaxed and neutralized hair, the conditioning composition (A4) comprises at least one acid. A preferred embodiment of the third subject matter a further step d), the cleansed hair of step c) is contacted with a conditioning composition (A4) comprising at least one acid and at least one conditioning compound for a period of from about 1 to about 20 minutes, especially for from about 3 to about 5 minutes, the composition (A4) is rinsed from the hair and optionally, the hair is dried.

Suitable conditioning compounds and total amounts are those named with respect to composition (A3). In principle, all cosmetically acceptable acids can be used in composition (A4). However, the use of specific acids is favorable in terms of removal of multivalent metal salts. In this respect, it is thus preferred if that the at least one acid in composition (A4) is selected from the group of acetic acid, lactic acid, citric acid and their mixtures, preferably from acetic acid. The use of lactic acid in composition (A4) has been proven to be especially effective in removing remaining multivalent metal salts on chemically relaxed and neutralized hair, thus further improving the good cosmetic properties obtained after steps a) to c) of the inventive method.

In this respect, it is furthermore preferred if the at least one acid in composition (A4) is present in a total amount of from about 0.1 to about 25% by wt., preferably of from about 0.1 to about 5% by wt., more preferably of from about 0.5 to about 4% by wt., especially of from about 0.5 to about 2% by wt., based on the total weight of composition (A4). The aforementioned amounts of the at least one acid, especially lactic acid, have been proven to effectively remove the multivalent metal salts still present on relaxed hair after steps a) to c) of the inventive method. Moreover, these amounts ensure that the hair cuticulas, which have been opened by the highly basic relaxing composition (A1), are completely closed. Thus, the good cosmetic properties of relaxed hair obtained after steps a) to c) can be further improved.

The composition (A4) can comprise further components as described for composition (A3).

In order to fully remove any traces of multivalent metal ions on the hair, it is preferred if a further step e) is carried out. This further step e) is preferably carried out from about 1 to about 2 weeks after the inventive steps a) to d) since the relaxation reaction on the hair can continue to progress for 1 to 2 weeks even after removal of the relaxing composition (A1) from the hair. It is therefore preferred if in a further step e), the conditioned hair of step d) is contacted with a cleaning composition (A5) comprising at least one acid and at least one surfactant for a period of from about 1 to about 20 minutes, especially for about 3 to about 5 minutes, the composition (A5) is rinsed from the hair and the hair is dried. The composition (A5) can be the same as composition (A3) used in step c) or can be different from composition (A3) used in step c) of the inventive method. It is preferred if the composition (A5) is different from the composition (A3) used in step c) of the inventive method.

In principle, all cosmetically acceptable acids can be used in composition (A5). However, the use of specific acids is favorable in terms of removal of the multivalent metal salts. In this respect, it is thus preferred if the at least one acid in composition (A5) is selected from the group of acetic acid, lactic acid, citric acid and their mixtures, preferably from acetic acid. The use of lactic acid in composition (A5) has been proven to be especially effective in removing multivalent metal salts still present on the hairs after steps a) to d), thus further increasing the good cosmetic properties obtained after steps a) to d) of the inventive method.

In this respect, it is furthermore preferred if the at least one acid in composition (A5) is present in a total amount of from about 0.1 to about 25% by wt., preferably of from about 0.1 to about 5% by wt., more preferably of from about 0.5 to about 4% by wt., especially of from about 0.5 to about 2% by wt., based on the total weight of composition (A5). The aforementioned amounts of the at least one acid, especially lactic acid, have been proven to effectively remove the multivalent metal salts still present on relaxed hair after steps a) and d) of the present disclosure, thus further improving the good cosmetic properties of relaxed hair obtained after steps a) to d).

The composition (A5) can comprise further components as described for composition (A3).

What was said above with respect to the treatment agents and the use of the chelating agents of formula (I) as contemplated herein applies, mutatis mutandis, with respect to further preferred embodiments of the method as contemplated herein.

In order to prevent unwanted reaction of components of the compositions (A1) to (A5) used in the inventive process, these compositions are preferably contained in separate containers. A fourth subject matter of the present disclosure is thus a kit of parts, comprising

a) at least one first container (C1), comprising a hair relaxing composition (A1) containing at least one multivalent metal hydroxide base in an amount sufficient to relax at least part of the keratinic fibers,
b) at least one second container (C2), comprising an inventive hair treatment agent (A2), and
c) at least one third container (C3), comprising an aqueous cleansing agent (A3) containing at least one surfactant and at least one acid and having a pH from about 1 to about 5.

As described with regard to the third subject matter, the relaxing composition is preferably prepared by mixing a composition containing a multivalent metal hydroxide with a composition containing a guanidine salt. Thus, a preferred embodiment of this subject matter kit further comprises at least one fourth container (C4), comprising at least one guanidinium salt, especially guanidinium carbonate.

The inventive kit can additionally comprise further containers for the conditioning composition (A4) and the cleaning composition (A5), which can be used after steps a) to c) of the inventive method. It is therefore preferred if the kit further comprises at least a fifth container (C5), comprising a conditioning composition (A4) containing at least one acid and at least one conditioning compound.

Moreover, it is preferred if the kit further comprises at least a sixth container (C6), comprising a cleaning composition (A5) containing at least one acid and at least one surfactant.

What was said above with respect to the treatment agents, the use of the chelating agents of formula (I) and the method as contemplated herein applies, mutatis mutandis, with respect to further preferred embodiments of the kit as contemplated herein.

### EXAMPLES

To demonstrate the effectiveness of the treatment agent, the use of chelating agents of formula (I) and method of the present disclosure, the following compositions were prepared. The weight percentage listed in each of the following compositions represents the amount of each ingredient present in the composition.

Relaxer cream containing multivalent metal hydroxide, especially calcium hydroxide

| | Amount in % by wt. |
|---|---|
| Petrolatum | 12.5 |
| Paraffinum Liquidum (Mineral Oil) | 9.4 |
| Multivalent metal hydroxide | 32 |
| Cetearyl alcohol | 7.8 |
| Prunus Amygadalus Dulcis | 5.2 |
| (Sweet Almond) Oil | |
| Oleth-10 | 1.9 |
| Ceteareth-20 | 1.9 |
| Panthenol | 0.72 |
| Calcium carbonate | 0.21 |
| Hydrolyzed keratin | 0.21 |
| Water | Ad 100 |

Activator containing guanidine salt, especially guanidine carbonate:

| | Amount in % by wt. |
|---|---|
| Guanidine salt | 10.2 |
| Carbomer | 2.0 |
| Hordeum Vulgare Seed Extract | 1.0 |
| Water | Ad 100 |

Inventive treatment composition (A2):

| | Amount in % by wt. |
|---|---|
| Chelating agent of formula (I) ¹⁾ | 3.0 |
| Diol ²⁾ | 0.15 |
| Triol ³⁾ | 0.18 |
| Cocamdiopropyl betaine | 4.1 |
| Polysorbate 20 | 2.0 |
| Panthenol | 1.5 |
| PEG-12 Dimethicone | 0.83 |
| Sodium chloride | 0.74 |
| PEG-12 Allyl Ether | 0.15 |
| PEG-12 | 0.07 |
| Water | Ad 100 |

¹⁾ a preferred chelating agent of formula (I) is the tetrasodium salt of N,N-bis(carboxymethyl)-L-glutamic acid,
²⁾ a preferred diol is propane-1,2-diol
³⁾ a preferred triol is glycerine

Cleaning agent (A3):

| | Amount in % by wt. |
|---|---|
| Surfactant ¹⁾ | 23.3 |
| Sodium Chloride | 4.2 |
| Petrolatum | 2.6 |
| Dimethicone | 1.2 |
| Acid ²⁾ | 1.2 |
| Polyquaternium-10 | 0.68 |
| Sodium benzoate | 0.52 |
| Panthenol | 0.39 |
| Guar Hydroxypropyltrimonium Chloride | 0.33 |
| Coconut Alcohol | 0.31 |
| CI 77891 | 0.20 |
| PEG-12 Allyl Ether | 0.08 |
| Isopropyl Alcohol | 0.07 |
| Mica | 0.06 |
| PEG-12 | 0.04 |
| Water | Ad 100 |

¹⁾ a preferred surfactant is a mixture of 13% by wt. Sodium Trideceth Sulfate, 6% by wt. cocamide MEA, 4% by wt. sodium lauroamphoacetate and 0.3% by wt. Behentrimonium chloride
²⁾ a preferred acid is lactic acid

A relaxing composition (A1) was prepared by mixing the relaxer cream and the activator in a weight ratio of 4:1 and applying the mixture on curly dry keratinic fibers. After 20 minutes at 23° C., the relaxing composition was rinsed from the hair. An inventive treatment composition (A2) was then applied to the wet hair and left for 3 minutes at 23° C. before rinsing the treatment composition from the hair. This hair was then treated with an aqueous cleaning composition (A3) for 3 minutes at 23° C. After rinsing the cleaning composition from the hair, the hair was blow dried and evaluated by 5 hair dressers with regard to hair shine and dryness of hair. Treatment of the hair with compositions (A1) and (A2) resulted in relaxed hair having a higher hair shine and less dryness of hair as compared to treatment of hair with composition (A1). If the hair was additionally treated with composition (A3), the hair shine was increased and the hair dryness was further reduced as compared to a treatment with composition (Al) or compositions (A1) and (A2).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Treatment agent for keratinic fibers, comprising in a cosmetically acceptable carrier,
a) at least one chelating agent of formula (I) 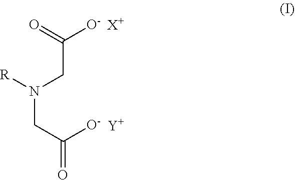
wherein
R is a C₁₋₁₀ alkyl group, a C₁₋₁₀ acyl group or a C₂₋₁₀ dicarbonyl group,
X⁺ and Y⁺ are, independently from each other, physiologically acceptable cations,
b) at least one diol and
c) at least one triol.

2. Hair treatment agent according to claim 1, wherein the residue R in formula (I) is a C₂₋₈ dicarbonyl group.

3. Hair treatment agent according to claim 1, wherein the residue R in formula (I) is a *—CHR¹—(CH₂)ₓ—COO⁻X⁺ group, wherein R¹ is a *—(CH₂)_{y}—COO⁻Y⁺ group, x is a number from 1 to 4, y is a number from 0 to 3 and X⁺ and Y⁺ are, independently from each other, physiological acceptable cations.

4. Hair treatment agent according to claim 1, wherein X⁺ and Y⁺ in formula (I) are independently H⁺, Li⁺, Na⁺, K⁺ or NH₄⁺.

5. Hair treatment agent according to claim 1, wherein the at least one chelating agent of formula (I) is the tetrasodium salt of N,N-bis(carboxymethyl)-L-glutamic acid.

6. Hair treatment agent according to claim 1, wherein the agent comprises the at least one chelating agent of formula (I), in a total amount of from about 0.1 to about 25% by wt.

7. Hair treatment agent according to claim 1, wherein the diol is a C₂₋₁₀ alkyl diol.

8. Hair treatment agent according to claim 1, wherein the agent comprises the at least one diol, in a total amount of from about 0.01 to about 20% by wt.

9. Hair treatment agent according to any claim 1, wherein the triol is a C₂₋₁₀ alkyl triol.

10. Hair treatment agent according to claim 1, wherein the agent comprises the at least one triol, in a total amount of from about 0.01 to about 5% by wt.

11. Hair treatment agent according to claim 1, wherein the agent comprises a weight ratio of the at least one diol b) to the at least one triol c) from about 10:1 to about 1:10.

12. Hair treatment agent according to claim 1, wherein the agent has a pH value from about 4 to about 10.

13. (canceled)

14. A method for relaxing keratinic fibers, the method comprising the sequential steps of:
a) contacting keratinic fibers, with a hair relaxing composition (A1) comprising at least one multivalent metal hydroxide base in an amount sufficient to relax at least part of the keratinic fibers for a period of from about 1 to about 20 minutes, and rising the composition (A1) from the hair to provide alkaline, chemically-relaxed hair;
b) contacting the alkaline, chemically-relaxed hair of step a) with a treatment agent (A2) according to claim 1 for a period of from about 1 to about 20 minutes and rinsing the composition (A2) from the hair; and
c) contacting the chemically-relaxed hair of step b) with an aqueous cleansing agent (A3) comprising at least one surfactant and at least one acid and having a pH from 1 to 5 for a period of from about 1 to about 20 minutes, rinsing the composition (A3) from the hair and optionally drying the hair.

15. Kit of parts, comprising
a) at least one first container (C1), comprising a hair relaxing composition (A1) comprising at least one multivalent metal hydroxide base in an amount sufficient to relax at least part of the keratinic fibers,
b) at least one second container (C2), comprising a hair treatment agent (A2) according to claim 1, and
c) at least one third container (C3), comprising an aqueous cleansing agent (A3) comprising at least one surfactant and at least one acid and having a pH from about 1 to about 5.
